(19) United States
(12) Patent Application Publication
(10) Pub. No.: US 2024/0180408 A1
(43) Pub. Date: Jun. 6, 2024

(54) Ingestible Camera System and Related Methods

(71) Applicants: David Craig-Lloyd White, Princess Anne, MD (US); Christopher David Parke, Princess Anne, MD (US)

(72) Inventors: David Craig-Lloyd White, Princess Anne, MD (US); Christopher David Parke, Princess Anne, MD (US)

(21) Appl. No.: 18/074,483

(22) Filed: Dec. 4, 2022

(51) Int. Cl. A61B 1/04; A61B 1/00

(52) U.S. Cl. CPC A61B 1/041; A61B 1/00016; A61B 1/00036

(57) ABSTRACT

The present disclosure provides a novel endoscopic imaging capsule and related methods. The capsule comprises a controller which causes a camera module to capture and transmit images while still inside a patient's gastrointestinal tract, transferring them to a predesignated IP address that is accessible by external devices, allowing the images to be accessed much faster. The capsule also has a rectangular profile, with lighting elements arranged to emit light orthogonally to the rectangular profile, providing a brighter, more focused image. It can also be encased in a gelatinous film to facilitate easier swallowing.

### FIELD OF INVENTION

The present invention relates generally to medical apparatus. More specifically, the present invention relates to an ingestible camera apparatus configured to capture and transmit images of internal organs.

### BACKGROUND

Vessel diseases, digestive diseases and cancers are some of the biggest risk health risks in humans. Effective treatment of such diseases is hugely dependent on early identification and diagnosis.

Traditionally, endoscopies have been widely used for observing the digestive system in medical treatments. However, endoscopies cannot capture images in some portions of the digestive system, such as the small intestines. Furthermore, endoscopies are highly uncomfortable and invasive procedures for patients.

In order to resolve these problems, capsule endoscopies have been developed in the industry. These typically include swallowable electronic capsules which collect data and which transmit the data to a receiver system. One widely used device for imaging the gastro-intestinal (GI) tract is the Pillcam (TM) Capsule Endoscopy System sold by Given Imaging. This product produces a visible light video image of the interior wall of the GI tract as the capsule camera device is pushed through it.

While the Pillcam and similar technologies offer advantages over traditional endoscopies, they have several drawbacks. Firstly, the captured images cannot be immediately viewed. Medical professionals must wait until the capsule has passed through the patient's digestive tract at a natural speed, then send the capsule to a facility where the images can be extracted and analyzed. Secondly, the shaped of the capsules mean that the light emitted by the capsule is too spread out, and is insufficient to see some portions of the GI tract. Thirdly, the capsules are often large and uncomfortable for patients to swallow.

It is within this context that the present invention is provided.

### SUMMARY

The present disclosure provides a novel endoscopic imaging capsule and related methods. The capsule comprises a controller which causes a camera module to capture and transmit images while still inside a patient's gastrointestinal tract, transferring them to a predesignated IP address that is accessible by external devices, allowing the images to be accessed much faster. The capsule also has a rectangular profile, with lighting elements arranged to emit light orthogonally to the rectangular profile, providing a brighter, more focused image. It can also be encased in a gelatinous film to facilitate easier swallowing.

According to a first aspect of the present disclosure, there is provided an ingestible camera apparatus, comprising: a waterproof housing having a rectangular profile; a camera module configured to capture images of a surrounding environment of the housing; one or more lighting elements arranged adjacent to the camera module and configured to shine light in a direction orthogonal to the rectangular profile of the housing; a power source disposed within the housing; a wireless communications module disposed within the housing; and a controller disposed within the housing.

The controller is configured to: cause the camera module to capture one or more images; and package and transmit one or more of the stored images to a predesignated IP address via the wireless communications module.

In some embodiments, the housing is encased in a soluble gelatinous casing. The gelatinous casing may in some examples comprise a laxative.

In some embodiments, the apparatus further comprises a voltage amplifier coupling the power source to the controller and camera module.

In some embodiments, the controller is further configured to transmit the images in real-time as they are captured.

In some embodiments, the wireless communications module is configured to communicate via wi-fi.

In some embodiments, the power source is a rechargeable battery.

In some embodiments, the apparatus further comprises a computer-readable medium, and the controller is further configured to store one or more captured images within the computer-readable medium.

In some embodiments, the housing is partially transparent, and the camera module and lighting elements are disposed within the housing facing out of a transparent portion of the housing. The housing may be arranged to reflect and focus light from the lighting elements in a direction orthogonal to the rectangular profile. The housing may have rounded corners

In some embodiments, the predesignated IP address is an IP address for a medical platform that saves and makes available the transmitted images to medical professionals.

In some embodiments, the controller is configured to begin capturing images after a predetermined time has elapsed since the power source was activated. Alternatively, the controller may be configured to begin capturing images in response to a command received from an external device via the wireless communications module.

In some embodiments, after capturing a first image, the controller is configured to capture images continuously at predetermined time intervals.

In some embodiments, the apparatus is configured to run in either a standard power mode or a low power mode where less power is supplied to the lighting elements.

In some embodiments, the apparatus further comprises a power level indicator for the power source.

### BRIEF DESCRIPTION OF THE DRAWINGS

Various embodiments of the invention are disclosed in the following detailed description and accompanying drawings.

FIG. 1 illustrates an isometric view of an exterior housing of an example camera apparatus according to the present disclosure.

FIG. 2 illustrates an isometric view of a set of internal components of the example camera apparatus.

FIG. 3 illustrates a first isometric view of a camera module and microcontroller board of the example camera apparatus.

FIG. 4 illustrates a second isometric view of the camera module and microcontroller board of the example camera apparatus.

FIG. 5 illustrates a first isometric view of a voltage converter of the example camera apparatus.

FIG. 6 illustrates a second isometric view of the voltage converter of the example camera apparatus.

FIG. 7 illustrates an isometric view of a rechargeable battery of the example camera apparatus.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

### Definitions

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The terms “about” and “approximately” shall generally mean an acceptable degree of error or variation for the quantity measured given the nature or precision of the measurements. Typical, exemplary degrees of error or variation are within 20 percent (%), preferably within 10%, and more preferably within 5% of a given value or range of values. Numerical quantities given in this description are approximate unless stated otherwise, meaning that the term “about” or “approximately” can be inferred when not expressly stated.

It will be understood that when a feature or element is referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments.

The terminology used herein is for the purpose of describing particular embodiments only and is not intended to be limiting. As used herein, the singular forms “a,” “an” and “the” are intended to include the plural forms as well, unless the context clearly indicates otherwise.

Spatially relative terms, such as “under,” “below,” “lower,” “over,” “upper” and the like, may be used herein for ease of description to describe one element or feature's relationship to another when the apparatus is right side up.

The terms “first,” “second,” and the like are used herein to describe various features or elements, but these features or elements should not be limited by these terms. These terms are only used to distinguish one feature or element from another feature or element. Thus, a first feature or element discussed below could be termed a second feature or element, and similarly, a second feature or element discussed below could be termed a first feature or element without departing from the teachings of the present disclosure.

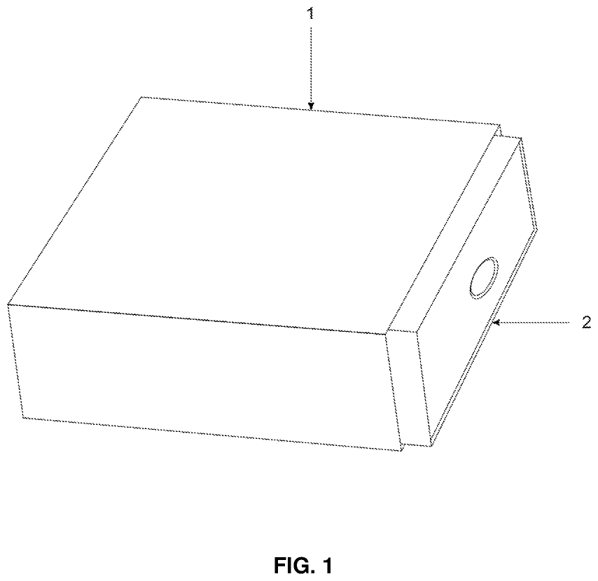

Referring to FIG. 1 an isometric view is shown of an exterior housing 2 of an example camera apparatus according to the present disclosure.

As can be seen, the housing 2 has a rectangular profile, which allows better focusing of light from the lighting elements (usually LEDs) on the gastrointestinal walls, providing brighter and thus better quality imaging. The housing 2 is waterproof and has rounded corners to prevent damaging the patient's tissue once ingested. One or more walls of the housing 2 may be transparent for the camera and LEDs.

In the present example the housing 2 is also wrapped in a gelatinous casing 1 to help swallowing of the camera apparatus be more palatable. The casing 1 is soluble such that it dissolves once ingested. It may comprise a small amount of laxative that, once dissolved, will help the draw water from the body and pass the camera apparatus through as quickly as possible.

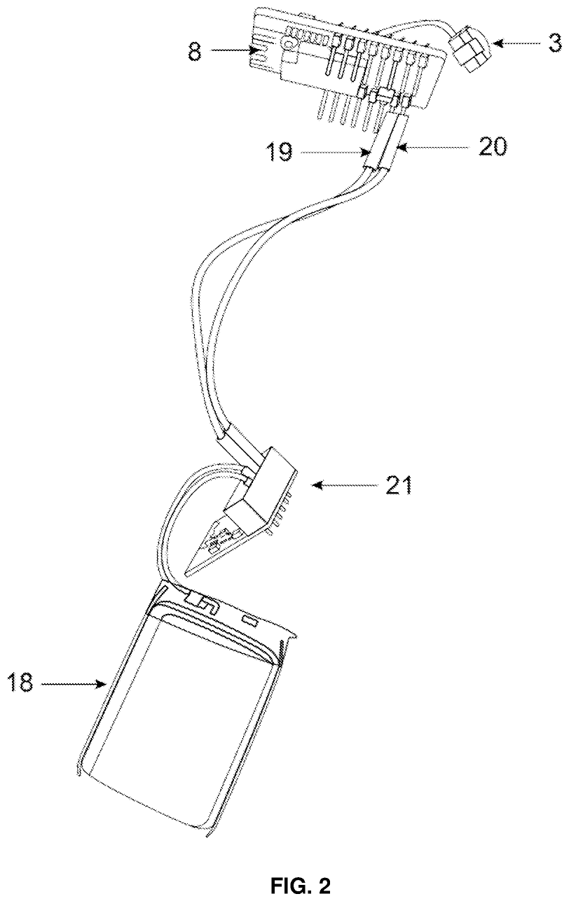

Referring to FIG. 2 an isometric view is shown of a set of internal components of the example camera apparatus.

The components include a 3.7V rechargeable lithium battery 18 which is wired, via a voltage amplifier board 21 that raises the voltage to 5V and a pair of ground 19 and positive 20 power source cables, to the controller board and camera module 3. A set of GPIO pins 8 handle the input and output of data and instructions from the controller board to the camera module 3.

All of the elements shown in FIG. 2 are housed inside the sealed housing of FIG. 1 and are thus protected from external conditions.

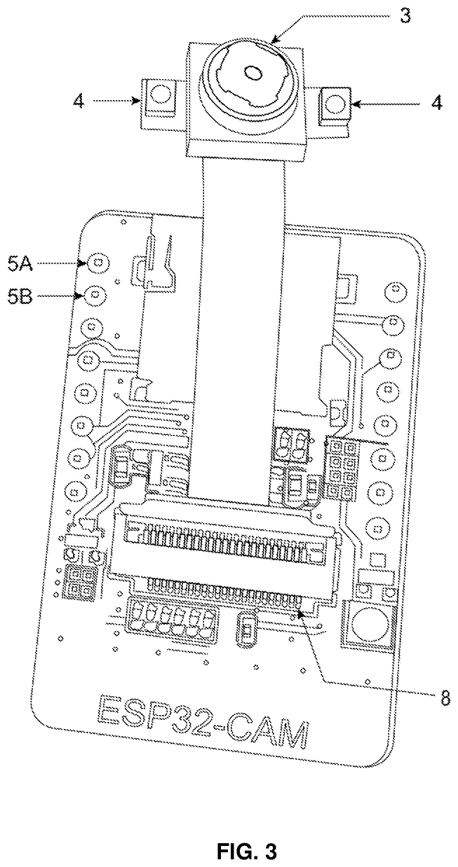

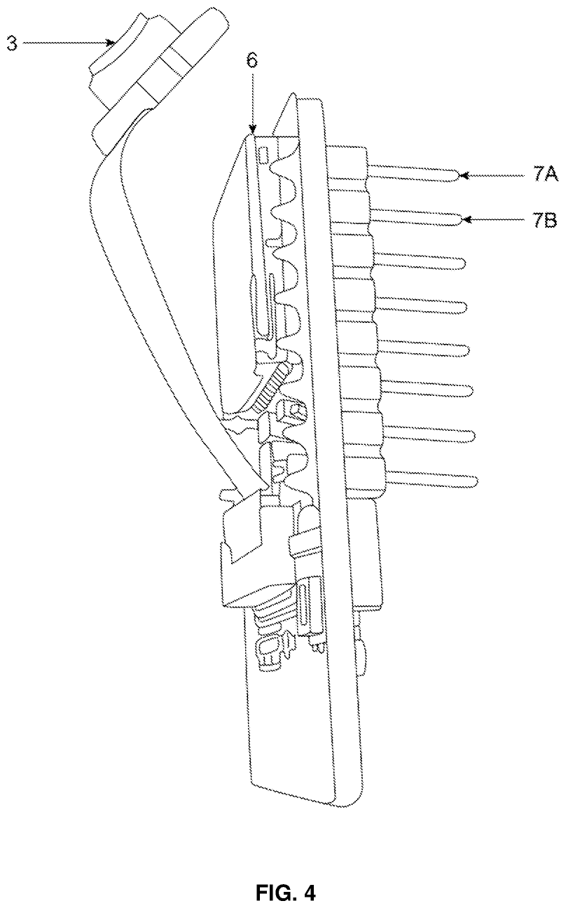

Referring to FIG. 3 and FIG. 4, first and second isometric views are shown of a camera module 3 and microcontroller board of the example camera apparatus.

As can be seen, the camera module 3 comprises a pair of LED lighting elements 4 either side for illuminating the environment of which images are being captured.

The microcontroller board actually has two different sets of power connection pins, a 5V input 5A and ground 5B on the front and a 3V input 7A and ground 7B on the back. The user can choose which to couple the power input to prior to ingestion based on how bright they would like the LEDs to illuminate the surroundings versus how long the camera module is expected to be operational for.

Once the power input for the camera apparatus has been chosen and connected the microcontroller will begin running a preinstalled code, communicating with the camera module 3 by way of the GPIO pins 8. A portion of the GPIO pins deliver power to the camera module 3, a second portion input commands to the camera module 3 (for example, instructing it when to capture an image) and a third portion of the pins receive output images captured by the camera module, the microcontroller then packaging and directing the data to a predesignated IP address via an onboard antenna to broadcast the information to an online platform where it will be saved and viewable through an app or web browser.

The images are often packaged and sent as they are captured, while moving through a patient's GI tract, but may also be sent at timed intervals, in which case they must be stored on a computer-readable medium of the camera apparatus until they are sent. In such examples, captured images are first stored on an SD card on the camera apparatus itself in slot 6.

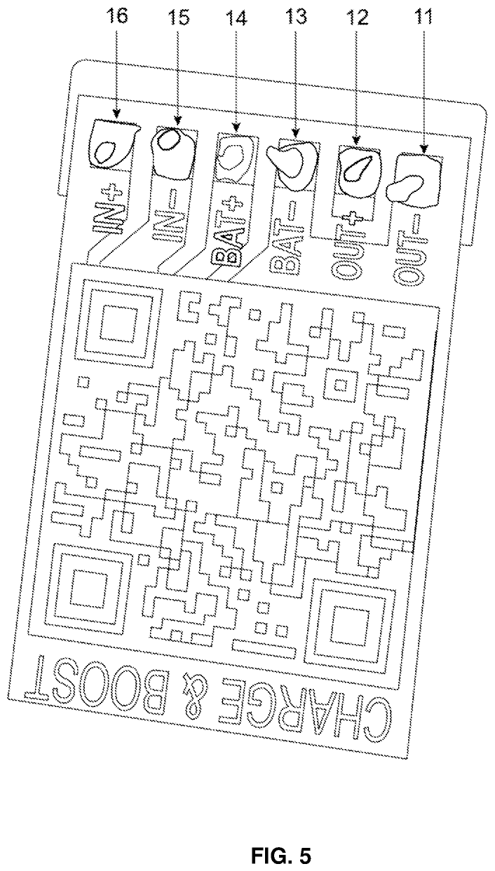

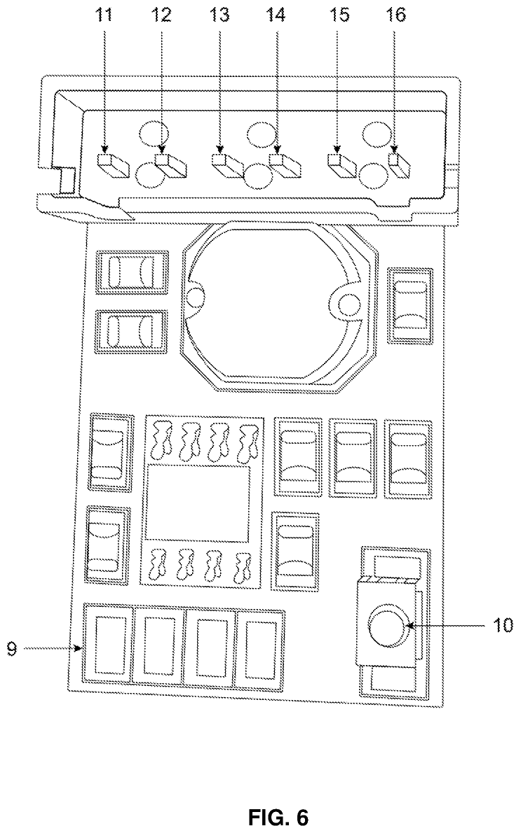

Referring to FIG. 5 and FIG. 6, first and second isometric views are shown of a voltage converter 21 of the example camera apparatus. This is a buck converter that adjusts the voltage from the power source to match the needs of the microcontroller and camera module.

The converter comprises a battery level indicator 9 and a reset control 10.

In the present example the voltage converter board amplifies the voltage from 3.7 volts up to 5 volts.

The 3.7V battery is plugged into the terminals on the voltage converter board labeled (IN+) 16 and (IN−) 15. The set of terminals labelled (OUT−) 12 and (OUT+) 11 then deliver the amplified 5 volt output to power the camera board.

The voltage converter board also has two terminals labeled (BAT+) 14 and (BAT−) 13. These terminals facilitate charging of the battery plugged into the (IN−) and (IN+) terminals.

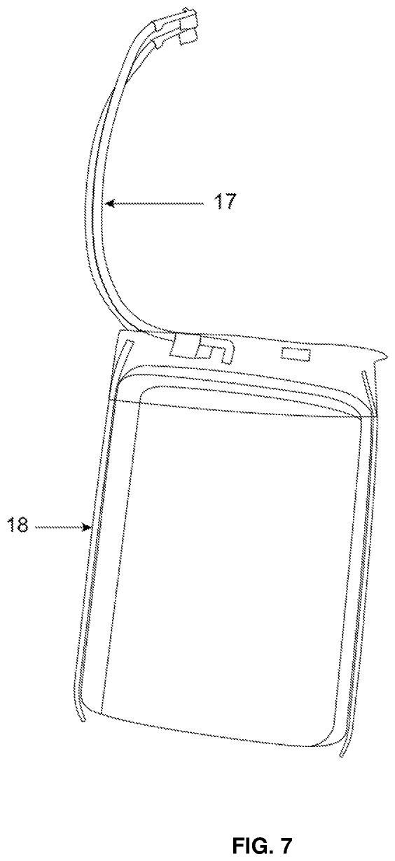

Referring to FIG. 7 an isometric view is shown of an example rechargeable battery 18 of the example camera apparatus, with the power cables 17 that connect it to the converter.

A controller can be any suitable type of small computer. A computer may be a uniprocessor or multiprocessor machine. Accordingly, a computer may include one or more processors and, thus, the aforementioned computer system may also include one or more processors. Examples of processors include sequential state machines, microprocessors, microcontrollers, graphics processing units (GPUs), central processing units (CPUs), application processors, digital signal processors (DSPs), reduced instruction set computing (RISC) processors, systems on a chip (SoC), baseband processors, field programmable gate arrays (FPGAs), programmable logic devices (PLDs), gated logic, programmable control boards (PCBs), and other suitable hardware configured to perform the various functionality described throughout this disclosure.

Additionally, the computer may include one or more memories. Accordingly, the aforementioned computer systems may include one or more memories. A memory may include a memory storage device or an addressable storage medium which may include, by way of example, random access memory (RAM), static random access memory (SRAM), dynamic random access memory (DRAM), electronically erasable programmable read-only memory (EEPROM), programmable read-only memory (PROM), erasable programmable read-only memory (EPROM), hard disks, floppy disks, laser disk players, digital video disks, compact disks, video tapes, audio tapes, magnetic recording tracks, magnetic tunnel junction (MTJ) memory, optical memory storage, quantum mechanical storage, electronic networks, and/or other devices or technologies used to store electronic content such as programs and data. In particular, the one or more memories may store computer executable instructions that, when executed by the one or more processors, cause the one or more processors to implement the procedures and techniques described herein. The one or more processors may be operably associated with the one or more memories so that the computer executable instructions can be provided to the one or more processors for execution. For example, the one or more processors may be operably associated to the one or more memories through one or more buses. Furthermore, the computer may possess or may be operably associated with input devices (e.g., a keyboard, a keypad, controller, a mouse, a microphone, a touch screen, a sensor) and output devices such as (e.g., a computer screen, printer, or a speaker).

The computer may advantageously be equipped with a network communication device such as a network interface card, a modem, or other network connection device suitable for connecting to one or more networks.

A computer may advantageously contain control logic, or program logic, or other substrate configuration representing data and instructions, which cause the computer to operate in a specific and predefined manner as, described herein. In particular, the computer programs, when executed, enable a control processor to perform and/or cause the performance of features of the present disclosure. The control logic may advantageously be implemented as one or more modules. The modules may advantageously be configured to reside on the computer memory and execute on the one or more processors. The modules include, but are not limited to, software or hardware components that perform certain tasks. Thus, a module may include, by way of example, components, such as, software components, processes, functions, subroutines, procedures, attributes, class components, task components, object-oriented software components, segments of program code, drivers, firmware, micro code, circuitry, data, and/or the like.

The control logic conventionally includes the manipulation of digital bits by the processor and the maintenance of these bits within memory storage devices resident in one or more of the memory storage devices. Such memory storage devices may impose a physical organization upon the collection of stored data bits, which are generally stored by specific electrical or magnetic storage cells.

The control logic generally performs a sequence of computer-executed steps. These steps generally require manipulations of physical quantities. Usually, although not necessarily, these quantities take the form of electrical, magnetic, or optical signals capable of being stored, transferred, combined, compared, or otherwise manipulated. It is conventional for those skilled in the art to refer to these signals as bits, values, elements, symbols, characters, text, terms, numbers, files, or the like. It should be kept in mind, however, that these and some other terms should be associated with appropriate physical quantities for computer operations, and that these terms are merely conventional labels applied to physical quantities that exist within and during operation of the computer based on designed relationships between these physical quantities and the symbolic values they represent.

It should be understood that manipulations within the computer are often referred to in terms of adding, comparing, moving, searching, or the like, which are often associated with manual operations performed by a human operator. It is to be understood that no involvement of the human operator may be necessary, or even desirable. The operations described herein are machine operations performed in conjunction with the human operator or user that interacts with the computer or computers.

It should also be understood that the programs, modules, processes, methods, and the like, described herein are but an exemplary implementation and are not related, or limited, to any particular computer, apparatus, or computer language. Rather, various types of general purpose computing machines or devices may be used with programs constructed in accordance with some of the teachings described herein. In some embodiments, very specific computing machines, with specific functionality, may be required.

In some embodiments, features of the computer systems can be implemented primarily in hardware using, for example, hardware components such as application specific integrated circuits (ASICs) or field-programmable gated arrays (FPGAs). Implementation of the hardware circuitry will be apparent to persons skilled in the relevant art(s). In yet another embodiment, features of the computer systems can be implemented using a combination of both general-purpose hardware and software

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the system and method have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

### LIST OF REFERENCE NUMERALS

1—Gelatinous Casing
2—Water proof Housing
3—Camera Module
4—LED Lighting elements
5A—5V power input
5B—Ground for 5V input
6—SD CARD slot
7A—3V power input
7B—Ground for 3V input
8—GPIO PINS
9—Battery level indicator
10—Reset control
11—Converter Voltage out ground pin
12—Converter Voltage out positive pin
13—Converter Battery ground pin
14—Converter Battery positive pin
15—Converter Voltage in ground pin
16—Converter Voltage in positive pin
17—Battery to Converter cable
18—Lithium Battery 3.7v
19—Converter to camera module Ground power source cable
20—Converter to camera positive power source cable
21—Voltage converter

What is claimed is:

1. An ingestible camera apparatus, comprising:
a waterproof housing having a rectangular profile;
a camera module configured to capture images of a surrounding environment of the housing;
one or more lighting elements arranged adjacent to the camera module and configured to shine light in a direction orthogonal to the rectangular profile of the housing;
a power source disposed within the housing;
a wireless communications module disposed within the housing; and
a controller disposed within the housing, the controller being configured to:
cause the camera module to capture one or more images; and
package and transmit one or more of the stored images to a predesignated IP address via the wireless communications module.

2. An ingestible camera apparatus according to claim 1, wherein the housing is encased in a soluble gelatinous casing.

3. An ingestible camera apparatus according to claim 2, wherein the gelatinous casing comprises a laxative.

4. An ingestible camera apparatus according to claim 1, wherein the apparatus further comprises a voltage amplifier coupling the power source to the controller and camera module.

5. An ingestible camera apparatus according to claim 1, wherein the controller is further configured to transmit the images in real-time as they are captured.

6. An ingestible camera apparatus according to claim 1, wherein the wireless communications module is configured to communicate via wi-fi.

7. An ingestible camera apparatus according to claim 1, wherein the power source is a rechargeable battery.

8. An ingestible camera apparatus according to claim 1, wherein the apparatus further comprises a computer-readable medium, and wherein the controller is further configured to store one or more captured images within the computer-readable medium.

9. An ingestible camera apparatus according to claim 1, wherein the housing is partially transparent, and wherein the camera module and lighting elements are disposed within the housing facing out of a transparent portion of the housing.

10. An ingestible camera apparatus according to claim 1, wherein the housing is arranged to reflect and focus light from the lighting elements in a direction orthogonal to the rectangular profile.

11. An ingestible camera apparatus according to claim 1, wherein the predesignated IP address is an IP address for a medical platform that saves and makes available the transmitted images to medical professionals.

12. An ingestible camera apparatus according to claim 1, wherein the housing has rounded corners.

13. An ingestible camera apparatus according to claim 1, wherein the controller is configured to begin capturing images after a predetermined time has elapsed since the power source was activated.

14. An ingestible camera apparatus according to claim 1, wherein the controller is configured to begin capturing in response to a command received from an external device via the wireless communications module.

15. An ingestible camera apparatus according to claim 1, wherein the controller is configured to capture images continuously at predetermined time intervals.

16. An ingestible camera apparatus according to claim 1, wherein the apparatus is configured to run in either a standard power mode or a low power mode where less power is supplied to the lighting elements.

17. An ingestible camera apparatus according to claim 1, wherein the apparatus further comprises a power level indicator for the power source.
